(19) United States
(12) Patent Application Publication
(10) Pub. No.: US 2026/0157969 A1
(43) Pub. Date: Jun. 11, 2026

(54) LYOPHILIZED COMPOSITIONS AND USES OF SAME

(71) Applicant: Boston Scientific Scimed, Inc., Maple Grove, MN (US)

(72) Inventors: Philip David Dorgan, Cork (IE); Katie J. O'Riordan, Cork (IE); Heather G. Cagney, Waterford (IE); Bhanu Prasanth Koppolu, Cary, NC (US); Eddy Mateo Flores, Limerick (IE)

(73) Assignee: Boston Scientific Scimed, Inc., Maple Grove, MN (US)

(21) Appl. No.: 19/407,171

(22) Filed: Dec. 3, 2025

Related U.S. Application Data
(60) Provisional application No. 63/730,781, filed on Dec. 11, 2024.

(51) Int. Cl. A61K 9/19; A61K 9/00; A61K 9/16

(52) U.S. Cl. CPC A61K 9/19; A61K 9/0019; A61K 9/1623; A61K 9/1647

(57) ABSTRACT

In some aspects the present disclosure provides sterile lyophilized compositions that comprise a block copolymer and a polyol, wherein the lyophilized composition is reconstitutable for injection. In other aspects the present disclosure provides methods of making such sterile lyophilized compositions, methods of forming sterile injectable fluid compositions by reconstituting such sterile lyophilized compositions, kits that comprise containers that contain such sterile lyophilized compositions and containers that contain sterile aqueous fluids, and medical procedures comprising forming sterile injectable fluid compositions from such sterile lyophilized compositions and administering the sterile injectable fluid compositions onto or into patient tissue.

### CROSS-REFERENCE TO RELATED APPLICATIONS

This application claims the benefit of U.S. Provisional Patent Application Ser. No. 63/730,781 filed on Dec. 11, 2024, the disclosure of which is incorporated herein by reference.

### FIELD

The present disclosure relates to lyophilized compositions, methods of making and using such lyophilized compositions, and injectable compositions that are formed from such lyophilized compositions. The lyophilized compositions are useful, for example, in conjunction with various medical procedures.

### BACKGROUND

In situ gel-forming compositions are attractive due to their minimally invasive delivery procedure, providing reduced healing time, reduced scarring, decreased risk of infection, and ease of delivery compared with surgically implanted materials. Such gel-forming compositions are especially useful for applications where the final form and shape of the compositions are either not important or are defined by the void or space into which they are injected or the surface onto which they are injected. Due to their ease of delivery, in situ gel-forming compositions are useful as embolic agents, tissue engineering compositions, and drug delivery depots, among other applications.

### SUMMARY

In various aspects, the present disclosure provides sterile lyophilized compositions that comprise at least one type of block copolymer and at least one type of polyol, wherein the lyophilized composition is reconstitutable for injection.

In some embodiments, the sterile lyophilized compositions the polyol is selected from a saccharide and a sugar alcohol. In some of these embodiments, the polyol is a saccharide. In some of these embodiments, the saccharide is selected from monosaccharides, disaccharides, trisaccharides and tetrasaccharides.

In some embodiments, which may be used in conjunctions with the above aspects and embodiments, the block copolymer is a thermosensitive block copolymer.

In some embodiments, which may be used in conjunctions with the above aspects and embodiments, the block copolymer is a diblock copolymer or a triblock copolymer.

In some embodiments, which may be used in conjunctions with the above aspects and embodiments, the block copolymer comprises one or more poly(C₁-C₄-alkylene oxide) segments. In some of these embodiments, the block copolymer further comprises one or more polyester segments. In some of these embodiments, the one or more polyester segments are selected from polyglycolic acid segments, polylactic acid, poly(lactic-acid-co-glycolic acid) segments, and polycaprolactone segments.

In some embodiments, which may be used in conjunctions with the above aspects and embodiments, the composition may further comprise one or more additional agents selected from imaging agents, colorants, tonicity adjusting agents, suspension agents, wetting agents, and pH adjusting agents.

In some embodiments, which may be used in conjunctions with the above aspects and embodiments, the compositions may further comprise a therapeutic agent. In some of these embodiments, the therapeutic agent is a protein-based biomolecular drug.

In some embodiments, which may be used in conjunctions with the above aspects and embodiments, compositions are contained in a vial having a septum.

In other aspects, the present disclosure pertains to methods of making sterile lyophilized compositions in accordance with any of the above aspects and embodiments, which comprise forming a solution that comprises the at least one block copolymer, the at least one polyol, the one or more additional agents, if any, and the therapeutic agent, if any, in a fluid medium, and lyophilizing the solution.

In some embodiments, the fluid medium is an aqueous fluid medium.

In other aspects, the present disclosure pertains to sterile injectable fluid compositions that comprise a mixture of a sterile lyophilized composition in accordance with any of the above aspects and embodiments and a sterile aqueous fluid.

In other aspects, the present disclosure pertains to methods of forming sterile injectable fluid compositions that comprise mixing a sterile lyophilized composition in accordance with any of the above aspects and embodiments and a sterile aqueous fluid.

In some embodiments, which may be used in conjunctions with the above aspects and embodiments, the sterile aqueous fluid is selected from water for injection, physiological saline, phosphate buffered saline, a contrast agent, and a sterile aqueous solution comprising a therapeutic agent. In some of these embodiments, the sterile aqueous fluid is a sterile aqueous solution comprising a therapeutic agent, and the therapeutic agent is a protein-based biomolecular drug.

In some embodiments, which may be used in conjunctions with the above aspects and embodiments, the sterile injectable fluid compositions are gellable upon injection into or application onto body tissues.

In other aspects, the present disclosure pertains to medical procedures that comprise administering to a subject a sterile injectable fluid composition in accordance with any of the above aspects and embodiments.

In some embodiments, the sterile injectable fluid compositions are administered by applying the sterile injectable fluid compositions onto body tissue of the subject or by injecting of the sterile injectable fluid compositions into body tissue of the subject.

In some embodiments, the administering comprises parenteral administration.

In some embodiments, which may be used in conjunctions with the above aspects and embodiments, the administering is performed using a syringe and/or a catheter.

In some embodiments, which may be used in conjunctions with the above aspects and embodiments, the administering is performed under image guidance.

In some embodiments, which may be used in conjunctions with the above aspects and embodiments, the sterile injectable fluid compositions form gels upon application of the sterile injectable fluid compositions onto body tissues of the subject or injection of the sterile injectable fluid compositions into body tissues of the subject.

In other aspects, the present disclosure pertains to methods of forming sterile injectable fluid compositions for administration, which comprise reconstituting a sterile lyophilized composition in accordance with any of the above aspects and embodiments in a sterile aqueous fluid.

In some embodiments, the sterile aqueous fluid is selected from water for injection, physiological saline, phosphate buffered saline, a contrast agent, and a sterile aqueous solution comprising a therapeutic agent. In some of these embodiments, the sterile aqueous fluid is a sterile aqueous solution comprising a therapeutic agent, and the therapeutic agent is a protein-based biomolecular drug.

In other aspects, the present disclosure pertains to kits that comprise a container that contains a sterile lyophilized composition in accordance with any of the above aspects and embodiments and a container of a sterile aqueous fluid.

In some embodiments, the sterile aqueous fluid is selected from water for injection, physiological saline, phosphate buffered saline, a contrast agent, and a sterile aqueous solution comprising a therapeutic agent.

In some embodiments, which may be used in conjunctions with the above aspects and embodiments, the kits further comprise a delivery device. In some of these embodiments, the delivery device comprises a syringe, a needle and optionally, a catheter.

### BRIEF DESCRIPTION OF THE DRAWINGS

The FIGURE schematically illustrates a delivery system for use in conjunction with the present disclosure.

### DETAILED DESCRIPTION

In some aspects, the present disclosure pertains to lyophilized compositions (also referred to as freeze-dried compositions) that comprise (a) one or more types of block copolymers and (b) one or more types of polyols. As used herein, a “polyol” is a molecule that contains two or more alcohol (≡C—O—H) groups.

Block copolymers for use in the compositions of the present disclosure include block copolymers that comprise one or more polyether segments, including poly(C₁-C₄-alkylene oxide) segments such as polyethylene oxide (PEO) segments, also referred to as polyethylene glycol (PEG) segments, polypropylene oxide (PPO) segments, and poly(ethylene oxide-co-propylene oxide) segments. The block copolymers may also comprise one or more polymer segments in addition to the one or more polyether segments, including one or more polyester segments such as polyglycolic acid (PGA) segments, polylactic acid (PLA) segments, including poly-l-lactic acid (PLLA) segments, poly-d-lactic acid (PDLA) segments and poly-d,l-lactic acid (PDLLA) segments, poly(lactic-acid-co-glycolic acid) (PLGA) segments, poly(F-caprolactone) (PCL) segments, and poly(lactic-acid-co-F-caprolactone) (PLCL) segments. A few specific examples of block copolymers for use in the present disclosure include A-B diblock copolymers, such as PLGA-PEG, PCL-PEG, PDLLA-PEG, PLCL-PEG and PEO-PPO, and A-B-A triblock copolymers such as PLGA-PEG-PLGA, PCL-PEG-PCL, PDLLA-PEG-PDLLA, PLCL-PEG-PLCL and PEO-PPO-PEO.

The weight average molecular weight (Mw) of the block copolymers for use in the present disclosure may range, for example, from 1,000 Da or less to 50,000 Da or more, for example, ranging anywhere from 1,000 Da to 2,000 Da to 5,000 Da to 10,000 Da to 20,000 Da to 50,000 Da (in other words ranging between any two of the proceeding numerical values).

In some embodiments, the block copolymers are thermosensitive block copolymers. Thermosensitive polymers include lower critical solution temperature (LCST) polymers, which can exhibit solubility in water at lower temperatures while becoming insoluble and forming a hydrogel with an increase in temperature, and upper critical solution temperature (UCST) polymers, which can exhibit solubility in water at higher temperatures while becoming insoluble and forming a hydrogel with a decrease in temperature.

Polyols are molecules that contain two or more alcohol (≡C—O—H) groups, typically three, four, five, six, eight, ten, twelve, fifteen, twenty or more alcohol groups. Illustrative polyols for use in the compositions of the present disclosure include straight-chained, branched and cyclic sugars and sugar alcohols and short polymers (including dimers, trimers, tetramers, pentamers, hexamers, heptamers, octamers, enneamers and decamers) of straight-chained, branched and cyclic sugars and sugar alcohols. Specific examples include monosaccharides such as fucose, ribose, arabinose, abequose, xylose, lyxose, rhamnose, galactose, galactosamine, N-acetylgalactosamine, galacturonic acid, dextrose, glucose, glucosamine, N-acetyl glucosamine, glucuronoic acid, iduronic acid, muramic acid, N-acetylmuramic acid, fructose, sorbose, mannose, pyranose, altrose, talose, tagatose, and pyranosides, disaccharides such as sucrose, lactose, maltose, isomaltose, trehalose, lactulose, and cellobiose, trisaccharides such as raffinose, nigerotriose, maltotriose, melezitose, maltotriulose, and kestose, tetrasaccharides such as lychnose, maltotetraose, nigerotetraose, nystose, sesamose, and stachyose, sugar alcohols such as glycerol, mannitol, sorbitol, inositol, xylitol, quebrachitol, threitol, arabitol, erythritol, pentaerythritol, dipentaerythritol, tripentaerythritol, adonitol, hexaglycerol, and dulcitol, starches, amylose, dextrins, cyclodextrins, as well as polyhydroxy crown ethers, and polyhydroxyalkyl crown ethers.

In some embodiments, the lyophilized compositions of the present disclosure contain from 50 wt % or less to 95 wt % or more of one or more types of block copolymers and between 5 wt % or less and 50 wt % or more of one or more types of polyols. For example, the lyophilized compositions of the present disclosure may contain anywhere from 50 wt % to 60 wt % to 70 wt % to 75 wt % to 80 wt % to 85 wt % to 90 wt % to 95 wt % (in other words, ranging between any two of the preceding values) of one or more types of block copolymers, among other values, and the lyophilized compositions of the present disclosure may contain anywhere from 5 wt % to 10 wt % to 15 wt % to 20 wt % to 25 wt % to 30 wt % to 40 wt % to 50 wt % of one or more types of polyols, among other values.

In some embodiments, the lyophilized compositions of the present disclosure may contain a molar ratio of one or more types of block copolymers to one or more types of polyols that ranges from 0.025:1 or less to 1:1 or more, for example ranging anywhere form 0.25:1 to 0.30:1 to 0.40:1 to 0.5:1 to 0.6:1 to 0.7:1 to 0.8:1 to 0.9:1 to 1:1, among other values.

The lyophilized compositions of the present disclosure may be formed using any method known in the lyophilization art. In various embodiments a solution is first formed that contains one or more types of block copolymers and one or more types of polyols dissolved in a suitable fluid medium, which may be, for example, an aqueous medium such as, for example, water, including ultrapure water and water for injection (WFI), saline, phosphate buffered saline (PBS), tris buffered saline or histidine buffer. The solution may contain, for example, from 10 wt % or less to 60 wt % or more of the combined amount of the one or more types of block copolymers and the one or more types of polyols, for example, ranging anywhere from 5 wt % to 10 wt % to 15 wt % to 20 wt % to 30 wt % to 40 wt % to 50 wt % to 60 wt %, among other values.

In some embodiments, the solution may be formed, for example, by (a) forming a first solution that contains one or more types of block copolymers dissolved in a suitable fluid medium, (b) forming a second solution that contains one or more types of polyols dissolved in a suitable fluid medium, and (c) combining the first and second solutions. In other embodiments, the solution may be formed, for example, by (a) forming an initial solution that contains one or more types of block copolymers dissolved in a suitable fluid medium, (b) adding one or more types of polyols to the initial solution and (c) dissolving the one or more types of polyols in the same. In further embodiments, the solution may be formed, for example, by (a) forming an initial solution that contains one or more types of polyols dissolved in a suitable fluid medium, (b) adding one or more types of block copolymers to the initial solution, and (c) dissolving the one or more types of block copolymers in the same.

The solution containing the one or more types of block copolymers and the one or more types of polyols is then lyophilized using any suitable method. Lyophilization, also known as freeze-drying, is a process that removes water from a frozen material by turning the ice directly into vapor without passing through a liquid phase. The solution is generally lyophilized by first freezing the solution and then lowering the pressure by placing the frozen material under a vacuum. In some embodiments, the resulting lyophilized composition is in the form of a particulate composition or a filamentous composition.

In various embodiments the lyophilized composition is subsequently reconstituted in a suitable aqueous fluid, as described in more detail below. In some embodiments, the one or more types of block copolymers retain their thermosensitive sol-gel characteristics upon reconstitution. In some embodiments, the lyophilized composition after reconstitution will have a solution-to-gel (sol-gel) transition in the range of 30° C. to 40° C.

In this regard, various block polymers, including thermosensitive triblock polymers, are in the form of highly viscous liquids which are difficult to handle and therefore limited in potential for further development and commercial execution. The present disclosure overcomes the viscous nature of such block copolymers by using polyols to stabilize the block copolymers and to create a lyophilized composition, such as a lyophilized particles or lyophilized filaments, which is more easily handled and reconstituted than a highly viscous liquid.

The lyophilized compositions of the present disclosure may be sterilized using any suitable method. For example, the compositions may be autoclaved while inside a reservoir, such as a syringe barrel, vial, or ampule by heating the mixture, for example, to a temperature of about 121° C. Alternatively or additionally, the compositions may be sterilized via sterile filtration, supercritical CO₂, gamma, x-ray or electron beam irradiation, or any other suitable technique.

In various embodiments, the lyophilized compositions of the present disclosure contain one or more agents (also referred to herein as “additional agents”) in addition to one or more types of block copolymers and one or more types of polyols. (Note that additional agents, including therapeutic agents, can also be added at the time of reconstitution of the lyophilized compositions as discussed further below.)

Additional agents include therapeutic agents. Examples of therapeutic agents include antithrombotic agents, anticoagulant agents, antiplatelet agents, thrombolytic agents, antibodies, anti-cancer drugs, antiproliferative agents, anti-inflammatory agents, hyperplasia inhibiting agents, anti-restenosis agents, steroids, anti-allergic agents, hemostatic agents, smooth muscle cell inhibitors, antibiotics, antimicrobials, anti-fungal agents, analgesics, anesthetics, immunosuppressants, growth factors, growth factor inhibitors, cell adhesion inhibitors, cell adhesion promoters, anti-angiogenic agents, cytotoxic agents, chemotherapeutic agents, immunotherapies including, checkpoint inhibitors, immune modulatory cytokines, T-cell agonists, Toll-like receptor agonists, oncolytic viral therapies and STING (stimulator of interferon genes) agonists, among others.

Examples of therapeutic agents among others include peptides, protein-based biomolecular drugs, including recombinant protein-based biomolecular drugs, such as antibodies, antibody-drug conjugates, enzymes, protein hormones, cytokines, transporters, metabolic regulators, coagulation factors, bone morphogenetic proteins, Fc fusion proteins, growth factors, interferons, interleukins, DAMPS (Damage-associated molecular pattern), and PAMPs (Pathogen-associated molecular pattern).

Saccharides have historically been known to increase protein stability in solution by preferential exclusion, improving the chemical potential of the native state by increasing the free energy of unfolding, thus preventing unfolding and damage of the protein entity (see, e.g., Li J, Wang H, Wang L, Yu D, Zhang X. ‘Stabilization effects of saccharides in protein formulations: A review of sucrose, trehalose, cyclodextrins and dextrans’. Eur J Pharm Sci. 2024 Jan. 1; 192:106625). Thus, incorporation of the saccharide as an excipient has been shown to retain protein structure and reduce degradation of the protein in solution.

Additional agents further include imaging agents, colorants, tonicity adjusting agents, suspension agents, wetting agents, and pH adjusting agents, including various buffer solutes.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd^{(III)}, Mn^{(II)}, Fe^{(III)} and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the lyophilized compositions of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including ^{99m}Tc, ²⁰¹Th, ⁵¹Cr, ⁶⁷Ga, ⁶⁸Ga, ¹¹¹In, ⁶⁴Cu, ⁸⁹Zr, ⁵⁹Fe, ⁴²K, ⁸²Rb, ²⁴Na, ⁴⁵Ti, ⁴⁴Sc, ⁵¹Cr and ¹⁷⁷Lu, among others, and (f) radiocontrast agents such as metallic particles, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of tonicity adjusting agents (beyond any tonicity provided by the one or more types of polyols and the one or more types of block copolymers) include inorganic salts (e.g., potassium chloride, sodium chloride, disodium hydrogen phosphate, potassium dihydrogen phosphate, etc.), among others.

In some embodiments, one or more additional agents may be included in the solution that contains the one or more types of block copolymers and the one or more types of polyols prior to the freeze-drying process. In some embodiments, one or more additional agents may be added at the time the lyophilized composition is reconstituted with an aqueous fluid as discussed below.

Regardless of when the one or more additional agents are combined with the one or more types of block copolymers and the one or more types of polyols, the one or more additional agents may be present, for example, in an amount up to 75 wt % or more, relative to the combined weight of the one or more types of block copolymers, the one or more types of polyols and the one or more additional agents, for example, ranging anywhere from 1 wt % or less to 2.5 wt % to 5 wt % to 10 wt % to 15 wt % to 20 wt % to 25 wt % to 30 wt % to 40 wt % to 50 wt % to 60 wt % to 75 wt %.

The lyophilized compositions of the present disclosure are generally stored and transported in a sterile form. The lyophilized compositions may be stored and/or transported, for example, in a vial, syringe barrel, ampoule, or other container.

In various embodiments, kits are provided, which include one or more containers of the lyophilized compositions as described herein also with additional components. For example, in addition to one or more containers of the lyophilized compositions as described herein, the kits may include one or more delivery devices for delivering the lyophilized compositions to a subject (e.g., a mammal, particularly, a human) such as syringe barrels, needles, catheters, or tubing sets. In some embodiments, the kits may comprise a lyophilized composition as described herein preloaded in a syringe barrel and/or in a container such as a vial or ampoule. Alternatively or in addition, the kits may be provided that include one or more accessory devices such as guidewires. Alternatively or in addition, the kits may be provided that include one or more containers of aqueous fluids (e.g. sterile water for injection, physiological saline, phosphate buffered saline, tris buffered saline, histidine buffer or contrast media). Alternatively or in addition, the kits may further comprise one or more additional agents, which may be selected, for example, from those described above, among others, which one or more additional agents may be provided in dry form or in solution form. Instructions, either as inserts or as labels, indicating quantities of the composition to be administered and/or guidelines for administration can also be included in the kits provided herein. In some embodiments, the instructions comprise instructions for performing one or more of the methods provided herein.

In various embodiments, the lyophilized compositions of the present disclosure are reconstituted with an aqueous fluid to form an injectable fluid composition, which is beneficially an injectable in situ gel-forming composition in some embodiments. Suitable aqueous fluids include ultrapure water, water for injection, saline, phosphate buffered saline, liquid contrast media, or aqueous fluids that contain one or more additional agents such as those described herein. For example, an aqueous liquid may be used to reconstitute a lyophilized composition as described herein in a vial. After the lyophilized composition is dissolved/dispersed in the aqueous liquid, the resulting injectable fluid composition is loaded into a syringe and injected into a subject via a catheter.

For cases where the aqueous fluid is an aqueous fluid that contains one or more additional agents, in some embodiments, the one or more additional agents are present as-received in an aqueous fluid that is used to reconstitute the lyophilized composition. In other embodiments, the one or more additional agents are present as-received in a solid or a fluid form, and a suitable aqueous medium (e.g., ultrapure water, water for injection, saline, phosphate buffered saline, etc.) may be used to dissolve, dispersed and/or dilute the one or more additional agents, thereby forming an aqueous fluid that is used reconstitute the lyophilized composition. For example, an aqueous medium may be injected from a syringe into a vial containing the one or more additional agents. After the one or more additional agents are dissolved, dispersed and/or diluted in the aqueous medium, the resulting aqueous fluid may be redrawn back into the same or another syringe and subsequently injected from the syringe into another vial containing a lyophilized composition as described herein. After the lyophilized composition is dissolved in the aqueous liquid, the resulting injectable fluid composition may be loaded into a syringe and subsequently injected into a subject via a catheter.

In various embodiments, the polyol acts as a tonicity modifier within the injectable fluid composition, along with any other added tonicity modifying components (e.g., present in tonicity modifying additional agents, normal saline, phosphate buffered saline, etc.). Adjusting the osmotic pressure of the final injectable fluid composition close to isotonic (˜290 mOsm/L) can prevent the injectable fluid composition from lysing red blood cells, thus minimizing tissue damage and patient discomfort during injection.

The injectable fluid compositions described herein can be administered by a variety of routes, depending upon the desired medical outcome. In some embodiments, the administering comprises injecting the injectable fluid composition. In some embodiments, the injectable fluid compositions are administered by parenteral administration. Parenteral administration includes intravenous, intraarterial, subcutaneous, intraperitoneal, or intramuscular injection or infusion. In some embodiments, the administering comprises an image guided procedure where computed tomography, fluoroscopy or ultrasound imaging is used to deliver the composition. In some embodiments, the administering comprises injecting the injectable fluid composition into the vascular system of a subject. In some embodiments, the administering comprises injecting the injectable fluid composition into a cancer of the subject or the vasculature supplying a cancer of the subject. In some embodiments, the administering comprises applying the injectable fluid composition onto tissue of a subject (e.g., by spraying). In some embodiments, the administering is performed using a catheter and/or a syringe.

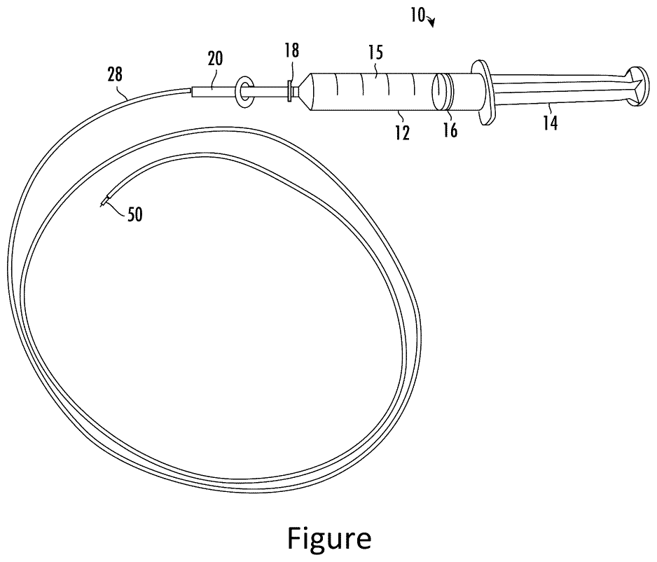

FIG. 1 illustrates an exemplary syringe 10 providing a reservoir for an injectable fluid composition 15 as discussed above. The syringe 10 may comprise a barrel 12, a plunger 14, and one or more stoppers 16. The barrel 12 may include a Luer adapter (or other suitable adapter/connector), e.g., at the distal end 18 of the barrel 12, for attachment to an injection needle 50 via a flexible catheter 29. The proximal end of the catheter 29 may include a suitable connection 20 for receiving the barrel 12. In other examples, the barrel 12 may be directly coupled to the injection needle 50. Upon depression of the plunger 14, the injectable fluid composition 15 is injected from the barrel 12, through the flexible catheter 29, and from the needle 50.

The injectable fluid compositions described herein can be administered to patients for achieving a number of medical outcomes.

The injectable fluid compositions described herein can be visualized (e.g., within a mammal) using any appropriate method during and/or after administration. For example, imaging techniques such as ultrasound, computed tomography, magnetic resonance imaging, and/or fluoroscopy can be used to visualize the injectable fluid compositions provided herein.

The injectable fluid compositions can be injected as a carrier of therapeutic agents in the treatment of diseases and cancers and the repair and regeneration of tissue, the injectable fluid compositions can be injected to provide spacing between tissues, the injectable fluid compositions can be injected for tissue augmentation or regeneration, the injectable fluid compositions can be injected as a filler or replacement for soft tissue, the injectable fluid compositions can be injected to provide mechanical support for compromised tissue, and/or the injectable fluid compositions can be injected as a scaffold, among other uses.

The injectable fluid compositions of the present disclosure may be used in a variety of medical procedures, including the following, among others: a procedure to implant a therapeutic-agent-containing depot comprising the injectable fluid composition, a procedure to implant a tissue regeneration scaffold comprising the injectable fluid composition, a procedure to implant a tissue support comprising the injectable fluid composition, a procedure to implant a tissue bulking agent comprising the injectable fluid composition, a tissue augmentation procedure comprising implanting the injectable fluid composition, a procedure to embolize tissue, including benign tumors, malignant tumors and other abnormal tissue, a procedure to control bleeding, a procedure to introduce the injectable fluid composition between a first tissue and a second tissue to space the first tissue from the second tissue.

The injectable fluid compositions may be injected in conjunction with a variety of medical procedures including the following: injection between the prostate or vagina and the rectum for spacing in radiation therapy for rectal cancer, injection between the rectum and the prostate for spacing in radiation therapy for prostate cancer, subcutaneous injection for palliative treatment of prostate cancer, transurethral or submucosal injection for female stress urinary incontinence, intra-vesical injection for urinary incontinence, uterine cavity injection for Asherman's syndrome, submucosal injection for anal incontinence, percutaneous injection for heart failure, intra-myocardial injection for heart failure and dilated cardiomyopathy, trans-endocardial injection for myocardial infarction, intra-articular injection for osteoarthritis, spinal injection for spinal fusion, and spine, oral-maxillofacial and orthopedic trauma surgeries, spinal injection for posterolateral lumbar spinal fusion, intradiscal injection for degenerative disc disease, injection between pancreas and duodenum for imaging of pancreatic adenocarcinoma, resection bed injection for imaging of oropharyngeal cancer, injection around circumference of tumor bed for imaging of bladder carcinoma, submucosal injection for gastroenterological tumor and polyps, visceral pleura injection for lung biopsy, kidney injection for type 2 diabetes and chronic kidney disease, renal cortex injection for chronic kidney disease from congenital anomalies of kidney and urinary tract, intravitreal injection for neovascular age-related macular degeneration, intra-tympanic injection for sensorineural hearing loss, dermis injection for correction of wrinkles, creases and folds, signs of facial fat loss, volume loss, shallow to deep contour deficiencies, correction of depressed cutaneous scars, perioral rhytids, lip augmentation, facial lipoatrophy, stimulation of natural collagen production.

The injectable fluid compositions may be injected for the permanent or temporary occlusion of blood vessels and thus may be useful for managing various diseases and conditions. For example, the injectable fluid compositions may be used for the controlled, selective obliteration of the blood supply to benign and malignant tumors including treating solid tumors such as renal carcinoma, bone cancer, brain cancer, liver cancer, breast cancer, prostate cancer, benign prostatic hyperplasia, esophageal cancer, colon cancer, endometrial cancer, bladder cancer, cancer of the uterus, uterine fibroids (leiomyoma), cancer of the ovary, lung cancer, sarcoma, pancreatic cancer, and stomach cancer. The idea behind this treatment is that the flow of blood, which supplies nutrients to the tumor, will be blocked causing it to shrink. Embolization may be conducted as an enhancement to chemotherapy or radiation therapy. Treatment may be enhanced in the present disclosure by including a therapeutic agent (e.g., antineoplastic/antiproliferative/anti-miotic agent, toxin, ablation agent, etc.) in the injectable fluid composition.

Injectable fluid compositions in accordance with the present disclosure may also be used to treat various other diseases, conditions and disorders, including treatment of the following: arteriovenous fistulas and malformations including, for example, aneurysms such as neurovascular and aortic aneurysms, pulmonary artery pseudoaneurysms, intracerebral arteriovenous fistula, cavernous sinus dural arteriovenous fistula and arterioportal fistula, varices, chronic venous insufficiency, varicocele, abscesses, pelvic congestion syndrome, gastrointestinal bleeding, renal bleeding, urinary bleeding, varicose bleeding, venous congestion disorder, hemorrhage, including uterine hemorrhage, and severe bleeding from the nose (epistaxis), as well as preoperative embolization (to reduce the amount of bleeding during a surgical procedure) and occlusion of saphenous vein side branches in a saphenous bypass graft procedure, among other uses. As elsewhere herein, treatment may be enhanced in the present disclosure by including a therapeutic agent in the injectable fluid composition.

Injectable fluid compositions in accordance with the present disclosure may be used further in tissue bulking applications, for example, as augmentative materials in the treatment of urinary incontinence, vesicourethral reflux, fecal incontinence, intrinsic sphincter deficiency (ISD) or gastro-esophageal reflux disease, or as augmentative materials for aesthetic improvement. For instance, a common method for treating patients with urinary incontinence is via periurethral or transperineal injection of a bulking material. In this regard, methods of injecting bulking agents commonly require the placement of a needle at a treatment region, for example, periurethrally or transperineally. The bulking agent is injected into a plurality of locations, assisted by visual aids, causing the urethral lining to coapt. In some cases, additional applications of bulking agent may be required. Treatment may be enhanced by including a therapeutic agent (e.g., proinflammatory agents, sclerosing agents, etc.) in the injectable fluid composition.

### Example

The ABA triblock copolymer used in the present example, PLGA-PEG-PLGA, is known in the literature for its ability to act as a drug delivery system, providing targeted drug delivery in vitro and in vivo (see Dutta, K. et al. (2020) ‘In situ forming injectable thermoresponsive hydrogels for controlled delivery of Biomacromolecules’, ACS Omega, 5(28), pp. 17531-17542; and Gao, Y. et al. (2010) ‘A thermo-sensitive PLGA-PEG-PLGA hydrogel for sustained release of Docetaxel’, Journal of Drug Targeting, 19(7), pp. 516-527). To achieve sustained drug release in vivo between 1-8 weeks, characteristics such as mechanical strength and molecular weight (Mw) of the ABA polymer hydrogel are preferably sufficiently great to create a platform by which a loaded biologic slowly diffuses from the hydrogel matrix (see Toews, P., & Bates, J. (2023). ‘Influence of drug and polymer molecular weight on release kinetics from HEMA and HPMA hydrogels’. Scientific Reports, 13(1), 16685). However, increases in polymer Mw are known to result in an inherent increase in polymer viscosity, leading to increased difficulty in reconstitution and handling (Marrucci, G., & Ianniruberto, G. (2000). ‘Molecular Theories of Polymer Viscosity’. In A. Fasano (Ed.), Complex Flows in Industrial Processes (pp. 3-24). Birkhauser Boston). The present Example demonstrates the ability of the present disclosure to mitigate the current handling issues seen with highly viscous ABA triblock copolymers by the creation of a lyophilized thermosensitive product that can be provided to the clinician in a stable divided form. Providing the lyophilized product in divided form improves storage of the hydrogel product, mitigating the ABA triblock copolymers circumvents need for specialized storage conditions. Additionally, this format improves the ease of handling, reconstitution, drug loading and administration to the patient.

To create the lyophilized product, an aqueous solution of (a) a saccharide, specifically sucrose (obtained from Sigma-Aldrich), dextran (obtained from), low acyl gellan gum (obtained from CP Kelco) or starch (obtained from Sigma-Aldrich), and (b) an ABA triblock copolymer, specifically PLGA-PEG-PLGA, Mw=5,000 Da, 9:1 lactide:glycolide ratio (obtained from Ashland Global, Wilmington, Delaware, USA) is first formed, after which the solution is lyophilized.

The ABA triblock copolymer was thick, sticky, and difficult to handle. Properties of the polyols or saccharides are shown in the following table, along with ten other candidates:

| | | | | | Glass | | | |
|---|---|---|---|---|---|---|---|---|
| | Mol Wt. | Physical | Melt/decomp | | transition | | Water | |
| Excipient | (g/mol) | appearance | point ° C. | | temperature | | Solubility | |
| Sucrose | 342.30 | white | 185-187° | C. | −34° | C. | 2.01 | g/ml |
| | | colorless | | | | | | |
| | | crystals | | | | | | |
| Dextran | 150,000 | White/almost | 53.75-54° | C. | −11° | C. | 30 | mg/ml |
| | | white powder | | | | | | |
| Starch | 50-5,000,000 | Fine white | 256-258° | C. | 227° C. (dry) | | 50 | mg/ml |
| | | powder | | | & 40-70° C. | | | |
| | | | | | (water | | | |
| | | | | | added) | | | |
| Gellan | 500,000 | white to off | 60-120° | C. | 42.98° | C. | highly | |
| Gum | | white powder | | | | | | |
| Trehalose | 378.33 | fine white | 97° C. | | 120° C. | | highly | |
| | | powder | (dihydrate) | | (anhydrous | | | |
| | | | | | amorphous | | | |
| | | | | | phase) | | | |
| Maltose | 360.312 | white powder | 119-121° | C. | N/A | | highly | |
| Mono- | | | | | (crystalline) | | | |
| hydrate | | | | | | | | |
| Mannitol | 182.17 | a white, | 166-1688° | C. | 13° | C. | 167 | mg/ml |

| | | odorless, | | | | | |
|---|---|---|---|---|---|---|---|
| | | crystalline | | | | | |
| | | powder | | | | | |
| Sorbitol | 182.17 | white or almost | Anhydrous | | −5° C. to | | 235 g/ |
| | | colorless, | form: | | −6° C. | | 100 g |
| | | crystalline, | 110-112.8° C. | | | | |
| | | hygroscopic | | | | | |
| | | powder | | | | | |
| Xylitol | 152.15 | white, granular | 92.0-96.08° | C. | −24° | C. | Freely soluble |
| | | solid. Xylitol is | | | | | |
| | | also | | | | | |
| | | commercially | | | | | |
| | | available in | | | | | |
| | | powdered form, | | | | | |
| | | and granular | | | | | |
| | | forms | | | | | |
| Malto- | 900-9,000 | nonsweet, | 240° | C. | 50° C. to | | Freely soluble |
| dextrin | | odorless, white | | | 150° C. | | |
| | | powder or | | | | | |
| | | granules | | | | | |
| Inulin | ~5000 | odorless white | 178.8° | C. | 124.85 | | Soluble in hot |
| | | powder with a | | | | | water; slightly |
| | | neutral to | | | | | soluble in cold |
| | | slightly | | | | | water |
| | | sweet taste. | | | | | |
| Fructose | 180.16 | Fructose occurs | ~102-105.8° | C. | 16-25° | C. | Good |
| | | as odorless, | | | | | solubility in |
| | | colorless | | | | | water at 20° C. |
| | | crystals or a | | | | | |
| | | white | | | | | |
| | | crystalline | | | | | |
| | | powder | | | | | |
| Dextrose | 198.17 | Dextrose occurs | 146.8° | C. | 57.85° | C. | Freely soluble |

| | | as odorless, | | | | |
|---|---|---|---|---|---|---|
| | | colorless | | | | |
| | | crystals or as a | | | | |
| | | white | | | | |
| | | crystalline or | | | | |
| | | granular | | | | |
| | | powder | | | | |
| Raffinose | 504.4 | Raffinose is a | 80-118.8° C. | 114.8° C. | 203 | mg/mL |

| | | |
|---|---|---|
| white | (anhydrous) | (amorphous) |
| crystalline | | |
| powder. | | |

To make the test solutions, a bulk solution of 15% w/w triblock copolymer solution was made up by combining 8.5 ml WFI water with 1.5 g polymer and agitating for 14 hours at 22° C. on shaker plate at 600 rpm. 1 ml of bulk solution was then sub-aliquoted into individual 20 ml vials. 150 mg of each saccharide was added to each vial in its solid form and the vials stirred overnight using a mechanical stirrer (at 22° C.), yielding a 1:1 saccharide:copolymer weight ratio for each saccharide. A visual assessment was completed, and saccharides which failed to solubilize in the triblock solution (i.e., starch) were discarded.

The solutions were then lyophilized. Vials were first pre-frozen at −20° C. overnight prior to lyophilization. The vials then were placed in a freeze dryer (Labconco FreeZone 12 Litre −84° C. Console Freeze Dryer, Labconco Corp., Kansas City, MO, USA) using the following settings: (a) vacuum timing: automatic, (b) PSI: 0.001 mBar, (c) temperature: −85° C. The freeze dryer was operated for 5 days.

Once time had elapsed, and the water content had been removed, the remaining lyophilized product was evaluated. The copolymer-sucrose product was found to be in the form of a crystalline powder. The copolymer-dextran product and the copolymer-gellan gum product were found to be in the form of filamentous structure. Because the copolymer-sucrose crystalline powder was found to display enhanced morphology, condition and handling ability, it was selected for further testing.

The copolymer-sucrose crystalline powder was reconstituted in 1 ml WFI and tested for thermoresponsive behavior by testing the reconstituted solution in a heating bath. The reconstituted solution was found to undergo a sol-gel transition at 38° C., indicating that the addition of the sucrose does not have an appreciable effect on the sol-gel transition of the copolymer. In particular, the reconstituted sucrose and polymer solution was injected into a microcentrifuge tube. In parallel, a water bath was heated using a hot plate until the temperature was confirmed to reach 38 degrees Celsius. At this point the microcentrifuge tube containing the sample was added to the heated bath. Gelling was confirmed both via opacity change of the solution, and visually by moving the gel from the microcentrifuge tube to a glass dish where it was seen to have clearly undergone phase transition.

In addition, bulk solutions of 15% w/w copolymer were made up as described above by combining either 8.5 ml WFI water or 8.5 ml PBS with 1.5 g of the copolymer. Then, sucrose in an amount of 150 mg, 75 mg, 25 mg, or 10 mg was added to vials containing 1 ml of WFI bulk solution. Sucrose in an amount of 150 mg, 75 mg, 25 mg, or 10 mg was also added and to vials containing 1 ml of PBS bulk solution. The vials were stirred overnight as described above, resulting in solutions with the following copolymer:sucrose molar ratios: 1:15 (for 150 mg sucrose), 1:7.5 (for 75 mg sucrose), 1:2.5 (for 25 mg sucrose), and 1:1 (for 10 mg sucrose).

The solutions were then lyophilized as described above and examined under a digital microscope. At the lowest concentration (10 mg/ml), the sucrose failed to provide sufficient structural stability to create a powder form, suggesting a loading limit had been reached. At higher concentrations a powder was formed for both the WFI and PBS samples. Salts that are present in the PBS were also found to have a potential additive effect on crystallinity, allowing for lower sucrose loading, if desired.

In the present Example, lyophilization is used to stabilize the copolymer. Without wishing to be bound by theory, it is hypothesized that the copolymer/saccharide product is stabilized through one, any two or all three of the following mechanisms: (a) hydrogen bonding: hydrogen bonds are formed with the polymer through the hydroxyl groups in the saccharide, which stabilizes the structure during the drying process; (b) glass formation: the saccharide forms a glassy matrix when dried, which immobilizes the polymer molecules, preserving the polymer structure preventing degradation; and (c) molecular mobility reduction: on drying, a rigid matrix is formed with the saccharide, which limits polymer molecule movement, preventing degradation processes.

It is also believed that the saccharide and polymer form a solid dispersion (SD), which will likely increase the rate of solubility of the copolymer, in a similar manner to how an SD can increase drug solubility (see Poka, M. S., et al. (2023). ‘Sugars and Polyols of Natural Origin as Carriers for Solubility and Dissolution Enhancement’. Pharmaceutics, 15(11)). Because it is a polar molecule, a saccharide is typically highly soluble in water. When added to a solution, the saccharide has the effect of enhancing the dissolution of other ingredients. It is hypothesized that the hydrogen bonding of water with hydroxyl groups of the saccharide will facilitate greater dissolution. Therefore, the reconstitution time for triblock copolymers may be reduced.

Additionally, the saccharide may act as a lyoprotectant within the process, thus protecting the integrity of the ABA polymer (see Rao, V. A., et al., (2020). ‘A Comprehensive Scientific Survey of Excipients Used in Currently Marketed, Therapeutic Biological Drug Products’. Pharmaceutical Research, 37(10)).

Although various embodiments are specifically illustrated and described herein, it will be appreciated that modifications and variations of the present disclosure are covered by the above teachings and are within the purview of any appended claims without departing from the spirit and intended scope of the present disclosure.

1. A sterile lyophilized composition comprising a block copolymer and a polyol, wherein the lyophilized composition is reconstitutable for injection.

2. The sterile lyophilized composition of claim 1, wherein the polyol is selected from a saccharide and a sugar alcohol.

3. The sterile lyophilized composition of claim 2, wherein the polyol is a saccharide.

4. The sterile lyophilized composition of claim 3, wherein the saccharide is selected from monosaccharides, disaccharides, trisaccharides and tetrasaccharides.

5. The sterile lyophilized composition of claim 1, wherein the block copolymer is a thermosensitive block copolymer.

6. The sterile lyophilized composition of claim 1, wherein the block copolymer is a diblock copolymer or a triblock copolymer.

7. The sterile lyophilized composition of claim 1, wherein the block copolymer comprises one or more poly(C₁-C₄-alkylene oxide) segments.

8. The sterile lyophilized composition of claim 7, wherein the block copolymer further comprises one or more polyester segments.

9. The sterile lyophilized composition of claim 8, wherein the one or more polyester segments are selected from polyglycolic acid segments, polylactic acid, poly(lactic-acid-co-glycolic acid) segments, and polycaprolactone segments.

10. The sterile lyophilized composition of claim 1, further comprising a therapeutic agent.

11. The sterile lyophilized composition of claim 10, wherein the therapeutic agent is a protein-based biomolecular drug.

12. The sterile lyophilized composition of claim 1, wherein the sterile lyophilized composition is in the form of a particulate composition or a filamentous composition.

13. A method of making the sterile lyophilized composition of claim 1, comprising forming a solution that comprises the block copolymer and the polyol in a sterile aqueous fluid medium, and lyophilizing the solution.

14. A method of forming a sterile injectable fluid composition comprising reconstituting the sterile lyophilized composition of claim 1 in a sterile aqueous fluid.

15. A medical procedure comprising (a) forming a sterile injectable fluid composition by a process comprising reconstituting a sterile lyophilized composition comprising a block copolymer and a polyol in a sterile aqueous fluid and (a) administering the sterile injectable fluid composition onto or into tissue of a subject.

16. The medical procedure of claim 15, wherein the sterile injectable fluid composition forms a gel as a result of a change in temperature to body temperature upon administering the sterile injectable fluid composition.

17. The medical procedure of claim 15, wherein the administering comprises parenteral administration.

18. A kit comprising (a) a first container that contains a sterile lyophilized composition comprising a block copolymer and a polyol, wherein the lyophilized composition is reconstitutable for injection in a sterile aqueous fluid and (b) a second container that contains the sterile aqueous fluid.

19. The kit of claim 18, wherein the sterile aqueous fluid is selected from water for injection, physiological saline, phosphate buffered saline, a contrast agent, and a sterile aqueous solution comprising a therapeutic agent.

20. The kit of claim 18, wherein the kit further comprises a delivery device that comprises a syringe, a needle and optionally, a catheter.
